(19) United States
(12) Patent Application Publication
(10) Pub. No.: US 2009/0025125 A1
(43) Pub. Date: Jan. 29, 2009

(54) Diving goggles with defogging device

(76) Inventor: Wan-Chen Jou, Banciao City (TW)

Correspondence Address: PRO-TECHTOR INTERNATIONAL, 20775 Norada Court, Saratoga, CA, 95070-3018, US

(21) Appl. No.: 11/881,296

(22) Filed: Jul. 26, 2007

(51) Int. Cl. A61F 9/02

(52) U.S. Cl. 2/428; 2/426; 2/435

(57) ABSTRACT

A diving goggles with defogging device, including a flexible frame provided with an adjustable fastening strap that is used to secure the diving goggles to the face of a user; an exterior lens used to seal off sea water from flowing into the flexible frame is fixed to the extreme exterior of the flexible frame; an internal lens which forms an airtight interlayer with the exterior lens is fixed to an inner side of the flexible frame; and a heating element used to maintain temperature of the internal lens is disposed on the internal lens. Accordingly, the present invention is able to effectively prevent fogging from occurring because of the temperature difference between body heat from the user and the lenses of the goggles.

### BACKGROUND OF THE INVENTION

(a) Field of the Invention

The present invention provides diving goggles with a defogging device that is able to prevent fogging.

(b) Description of the Prior Art

The main reason for fogging of diving goggles Is because when diving, the external water temperature is usually low, whereas face temperature within the diving goggles of the diver is higher than that of the water temperature, thus, a temperature difference is formed between the lens of the diving goggles and the face, which, because of low temperature condensation, results in the heat radiating from the face condensing and forming small water droplets on the glass on the inside of the diving goggles

A defogging method of prior art involves the diver putting on the diving goggles and deliberately raising the front portion to flood the inside of the lens with sea water, the diving goggles are then replaced on the face after washing away the fog, after which an appropriate expiration through the nose eliminates the flooded sea water, thereby completing the prior art process to eliminate fogging. Hence, this series of actions is an unavoidable necessity experienced persons meticulously carry out, to say nothing of the uneasiness it brings to the many beginner divers. If the series of actions are ignored, of less importance will be the inability to enjoy the beautiful underwater scenery, and what the diver will see will be nothing more than blurred scenes, of critical importance will be the unclear line of sight of the diver, resulting in becoming separated from the diving group and becoming lost in the hazardous sea area.

Apart from the aforementioned prior art, various other types of preventive defogging methods can be carried out before entering the water, for example, spitting saliva onto the lens of the diving goggles and smearing it with the fingers, or applying a commercial chemical defogging agent, and so on. However, for a user, because of the poor effectiveness of adopting the saliva procedure, one flooding action washes the lens perfectly clean again, and whereas the chemical defogging agent provides slightly better effectiveness, the chemical defogging agent actually contains volatile substances that irritate the eyes, thus, if an excessive amount of the chemical defogging agent is applied to the lens, it can cause damage to the eyes. Furthermore, use of a fog remover agent merely slightly slows down the time for the lens of the diving goggles to fog up again.

### SUMMARY OF THE INVENTION

A primary objective of the present invention is to provide diving goggles with a defogging device that effectively prevent fogging from occurring because of the temperature difference between body heat from the user and the lenses of the goggles, thereby enabling the diving goggles to maintain good operating effectiveness.

To enable a further understanding of said objectives and the technological methods of the invention herein, a brief description of the drawings is provided below followed by a detailed description of the preferred embodiments.

### BRIEF DESCRIPTION OF THE DRAWINGS

FIG. 1 shows a preferred embodiment of diving goggles with defogging device of the present invention.

FIG. 2 shows relative relationship of a heating element and an internal lens according to the present invention

FIG. 3 shows another relative relationship between the heating element and the internal lens according to the present invention

FIG. 4 shows another preferred embodiment of diving goggles with defogging device of the present invention.

### DETAILED DESCRIPTION OF THE PREFERRED EMBODIMENTS

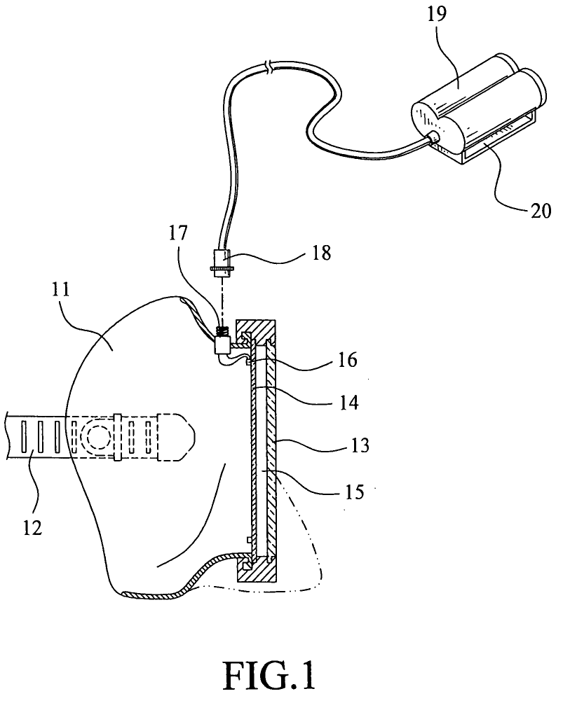

Referring to FIG. 1, which discloses a preferred embodiment of the present invention depicting diving goggles provided with a defogging device, comprising: A flexible frame 11 provided with an adjustable fastening strap 12 that is used to secure the diving goggles to the face of a user; an exterior lens 13 used to seal off sea water from flowing into the flexible frame 11 is fixed to the extreme exterior of the flexible frame 11,; an internal lens 14 forming an airtight interlayer 15 with the exterior lens 13 is fixed to an inner side of the flexible frame 11; and a heating element 16 used to maintain temperature of the internal lens 14 is disposed on the internal lens 14.

In a practical application of the present invention, the exterior lens 13 is made from tempered glass, and the heating element 16 is connected to a waterproof power supply socket 17 and disposed on the flexible frame 11. A waterproof electric connection 18 is connectively inserted into the waterproof power supply socket 17, and a battery pack holder 19, which provides a power source, is connected to the waterproof electric connection 18. For the convenience of portability, the diving goggles comprise a waist/shoulder belt ring 20 disposed on the battery pack holder 19, and which is used to fasten the battery pack holder 19 to the body of a user.

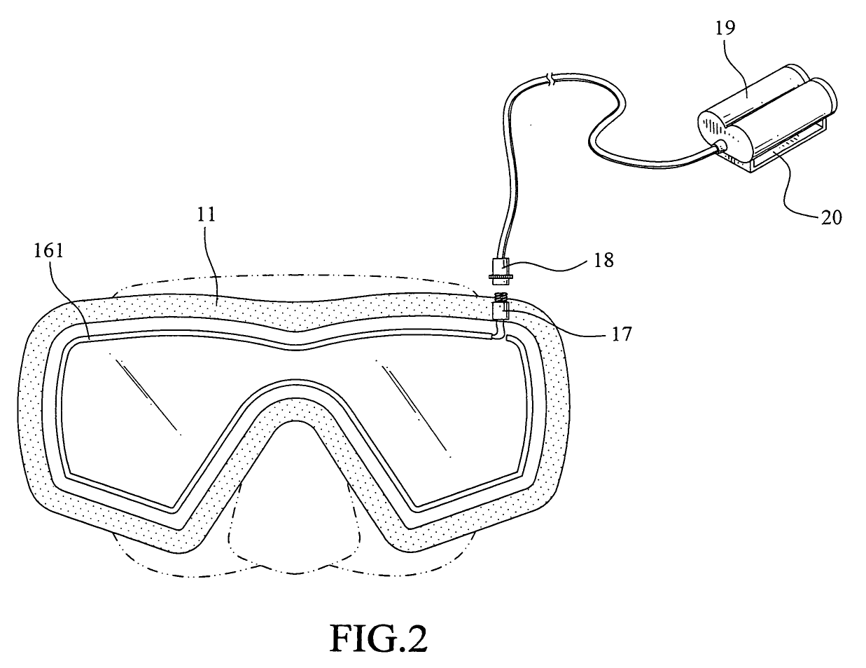

Referring to FIG. 2, which explicitly discloses the relative relationship between the heating element 16 and the internal lens 14, wherein the heating element 16 is a defogging electric heating wire 161 attached to a periphery of the internal lens 14. Direct current supplied by the battery pack holder 19 is used to provide power to the defogging electric heating wire 161, thereby causing the defogging electric heating wire 161 to emit slight heat and achieve a gentle heating effect. The defogging electric heating wire 161 can be attached to the lens using bonding technology similar to that used to attach a defogging device to the rear window of an automobile.

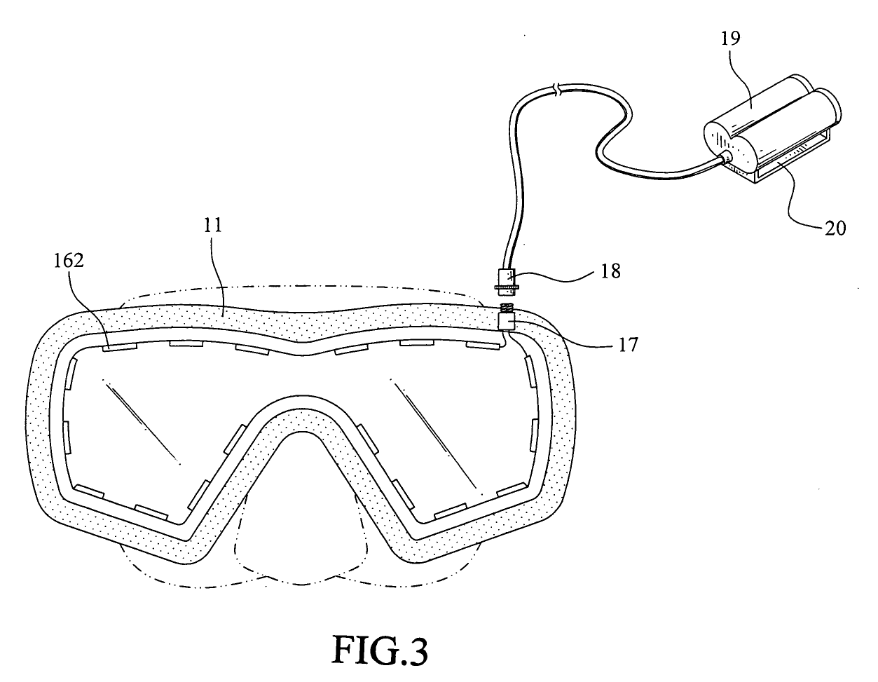

Referring to FIG. 3, which discloses another relative relationship between the heating element 16 and the internal lens 14, wherein a heating element 162 is a set of ceramic heating strips 162 which is attached to the periphery of the internal lens 14. The attached set of ceramic heating strips 162 uses ceramic heating strips designed with appropriate impedance and current effect to produce heat, thereby achieving heating effectiveness.

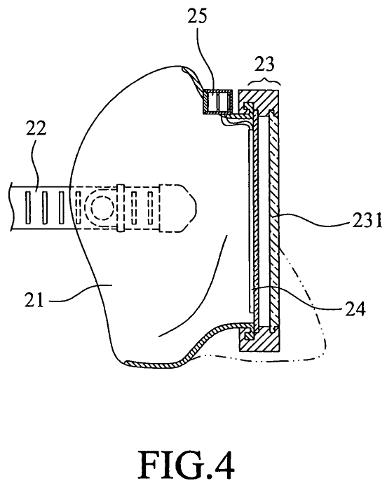

Referring to FIG. 4, which discloses another embodiment of the diving goggles having a defogging device, structured to comprise a flexible frame 21 provided with an adjustable fastening strap 22 that is used to secure the diving goggles to the face of a user; and a multilayer lens set 23 used to outwardly seal off sea water from flowing into the flexible frame 21 is fixed to the flexible frame 21. A heating element 24 used to maintain temperature of the inner side of the multilayer lens set 23 is located on an inner side of the multilayer lens set 23.

In a practical application of the embodiment, an exterior lens 231 made from tempered glass is located on an extreme exterior of the multilayer lens set 23, and the heating element 24 is a transparent electroconductive film, which is an ITO transparent electroconductive film formed by using a chemical vapor deposition CVD film method. A miniature battery 25 disposed on the flexible frame 21 is used to transmit voltage to the transparent electroconductive film, thereby generating heat uniformly on the lens. It is of course understood that in order to accommodate the use environment of the diving goggles, the miniature battery 25 can be replaced with a sea water battery, thereby facilitating use of the diving goggles in sea water.

A layer of relatively thin heat insulating glass is further disposed within the first layer of tempered glass of the diving goggles of the present invention, thereby enabling using the sealed space between the two layers of glass to isolate the temperature difference between the inside and outside. The principle behind such a configuration is that when heat from the face is outwardly dissipated through the heat insulating glass, it meets with the sealed interlayer which prevents conduction of the heat by means of air convection and a solid medium, thereby causing the heat to fade away. In comparison, when in contact with sea water, the tempered glass prevents the colder temperature of the sea water from being conducted inward. Hence, the two lenses simultaneously prevent mutual interference of different respective temperatures, and thereby form an effective and good isolation of the temperature difference. It is of course understood that the heating method for the inner layer heat insulating glass of the diving goggles of the present invention is not limited to the aforementioned three methods. Combining the various concepts of the present invention, various grades of the defogging diving goggles can be fabricated in accordance with cost considerations, thereby providing the user with more choices. The direct current battery pack is designed in principle to be attached to a shoulder belt or waist belt, and a rubber tightening screw thread design is used between an extension line and the electrode connection point of the diving goggles to achieve firm, convenient and waterproof functionality.

As is apparent from the foregoing, structure of the diving goggles with defogging device provided by the present invention is simple, and is able to effectively prevent fogging from occurring because of the temperature difference between body heat from the user and the lenses of the diving goggles, thereby enabling the diving goggles to maintain good operating effectiveness.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A diving goggles with defogging device, comprising at least:
a flexible frame provided with an adjustable fastening strap that is used to secure a diving goggles to the face of a user;
an exterior lens fixed to the extreme exterior of the flexible frame and used to seal off sea water from flowing into the flexible frame;
an internal lens fixed to an inner side of the flexible frame, thereby forming an airtight interlayer with the exterior lens; and
a heating element disposed on the internal lens and used to maintain temperature of the internal lens.

2. The diving goggles according to claim 1, wherein the exterior lens is made from tempered glass.

3. The diving goggles according to claim 1, wherein the heating element is a defogging electric heating wire.

4. The diving goggles according to claim 3, wherein the defogging electric heating wire is attached to a periphery of the internal lens.

5. The diving goggles according to claim 1, wherein the heating element is a set of ceramic heating strips.

6. The diving goggles according to claim 5, wherein the ceramic heating strips are attached to the periphery of the internal lens.

7. The diving goggles according to claim 1, wherein the heating element is provided with:
a waterproof power supply socket disposed on the flexible frame;
a waterproof electric connection connectively inserted into the waterproof power supply socket; and
a battery pack holder connected to the waterproof electric connection and used to provide a power source.

8. The diving goggles according to claim 7, further comprising a waist/shoulder belt ring disposed on the battery pack holder, and used to fasten the battery pack holder to the body of a user.

9. The diving goggles according to claim 1, wherein the heating element is a transparent electroconductive film.

10. The diving goggles according to claim 9, wherein the transparent electroconductive film is an ITO transparent electroconductive film formed by using a chemical vapor deposition CVD film method.

11. A diving goggles with defogging device, comprising at least:
a flexible frame provided with an adjustable fastening strap that is used to secure a diving goggles to the face of a user; and
a multilayer lens set fixed to the flexible frame and used to outwardly seal off sea water from flowing into the flexible frame, wherein a heating element is located on an inner side of the multilayer lens set, and is used to maintain temperature of the inner side of the multilayer lens set.

12. The diving goggles according to claim 11, wherein the multilayer lens comprises an exterior lens made from tempered glass.

13. The diving goggles according to claim 11, wherein the heating element is a defogging electric heating wire.

14. The diving goggles according to claim 13, wherein the defogging electric heating wire is attached to a periphery of the multilayer lens.

15. The diving goggles according to claim 11, wherein the heating element is a set of ceramic heating strips

16. The diving goggles according to claim 15, wherein the ceramic heating strips are attached to the periphery of the multilayer lens.

17. The diving goggles according to claim 11, further comprising:
a waterproof power supply socket disposed on the flexible frame;
a waterproof electric connection connectively inserted into the waterproof power supply socket; and
a battery pack holder connected to the waterproof electric connection and used to provide a power source.

18. The diving goggles according to claim 17, further comprising a waist/shoulder belt ring disposed on the battery pack holder, and used to fasten the battery pack holder to the body of a user.

19. The diving goggles according to claim 11, wherein the heating element is a transparent electroconductive film.

20. The diving goggles according to claim 19, wherein the transparent electroconductive film is an ITO transparent electroconductive film formed by using a chemical vapor deposition CVD film method.
